(19) United States
(12) Patent Application Publication
(10) Pub. No.: US 2011/0047681 A1
(43) Pub. Date: Mar. 3, 2011

(54) PROTECTIVE EYEWEAR DEVICE WITH LATERAL EYE ACCESS

(76) Inventor: Arman Hedayat, Windermere, FL (US)

(21) Appl. No.: 12/546,718

(22) Filed: Aug. 25, 2009

(51) Int. Cl. A61F 9/02

(52) U.S. Cl. 2/435; 2/452

(57) ABSTRACT

Disclosed herein are protective eyewear device embodiments that enable lateral access to the eye while at the same time shielding the eye from other directions. The protective eyewear device embodiments are particularly suitable for water activities as they allow air flow between the user and the device, thereby avoiding fogging of the lens. As water eventually enters in between the user and the device, which ultimately interferes with the eyes of the user, the user can wipe the eyes via the lateral access without actually having to remove the protective eyewear device.

### FIELD OF THE INVENTION

The present invention relates to protective eyewear, particularly to sport goggles used to deflect water, air and other debris from the eye during water activities and allow a user to access the eye while in use.

### BACKGROUND OF THE INVENTION

Various protective eyewear devices are used to prevent particulate matter, liquid, and other debris from entering the eyes. Protective eyewear devices such as sports goggles are used in different sporting activities including both water and non-water sports. Sport goggles provide protection to the user during participation in various sports activities while simultaneously providing the user with a clear view of his surroundings.

Sport goggles must be constructed to prevent fogging of the lens during use so that the user maintains a clear vantage point while in use. Furthermore, access to the eye during use is preferable, particularly when goggles are used for water sports.

### SUMMARY

In one embodiment, the present invention provides a protective eyewear device for wearing on a face of a user. The device includes a frame body with a front side, rear side, a bottom portion with a bottom side, and a top portion with a top side. The frame also includes a first and a second lateral end on each lateral side of the frame, and the frame is configured such that the frame contacts the face except at each lateral end. Additionally, the device includes a transparent lens secured onto the front side of the frame with a securing member to hold the frame onto the user's face such that said rear side of the frame is in contact with the face. The securing member attaches onto the frame at the first and second lateral ends of the frame to create a lateral opening on both first and second lateral ends of the frame in order to form a gap between the face of the user and the device, allowing the user access to the eye during use of the device.

### BRIEF DESCRIPTION OF THE DRAWINGS

A more particular description of the invention briefly described above will be rendered by reference to specific embodiments thereof that are illustrated in the appended drawings. Understanding that these drawings depict only typical embodiments of the invention and are not therefore to be considered limiting of its scope, the invention will be described an explained with additional specificity and detail through the use of the accompanying drawings in which:

FIG. 1 depicts a front view of an exemplary embodiment of a protective eyewear device

FIG. 2 represents a cross-section view of the device shown in FIG. 1 along the plane 9-9.

FIG. 3 shows a side view of the embodiment shown in FIG. 1 illustrating the gap formed by the lateral opening on the side of the protective eyewear device.

FIG. 4 depicts a top view of an exemplary embodiment of a protective eyewear device.

FIG. 5 depicts a side rear view of the an embodiment of a protective eyewear device.

FIG. 6 shows an embodiment of a protective eyewear device that has a securing member that includes two separate bands.

FIG. 7 shows a front view of an alternative embodiment of a protective eyewear device.

FIG. 8 shows a top view of the embodiment shown in FIG. 7.

### DETAILED DESCRIPTION OF THE INVENTION

A common problem with most sports goggles which fit closely to the user's face is their tendency to fog during use. Most known types of sports goggles include an anti-fogging mechanism which enhances performance of the goggle during use. These anti-fogging mechanisms can often malfunction and most are typically unsuccessful in goggles used for water-related activities.

Another problem with goggles, as realized by the inventor, particularly associated with goggles used for water sports, is the accumulation of water between the lens and the face of the wearer. Most water-friendly goggles fail to prevent water from entering the user's eyes, particularly during activities where the impact of water to the face is significantly increased, as in wind surfing, wakeboarding, or jet skiing, among others. In cases where water does enter the protected-eye area of the goggle, it is held there by the portion of the goggle which secures the device to the user's face.

A further concern with goggles, particularly those used in water sports, is the inability of the user to access the protected eye area if necessary. In cases where a foreign element such as debris or water gets trapped in the eye-region of the goggle, and the goggle-structure itself prevents access to the eye, removal of the foreign element can be a challenge. When water is trapped inside the goggles during use, removal of water from the goggle structure, or ability to wipe the eye after exposure to water can be difficult with most known water-friendly goggles. This is especially true in the case of conducting water activities in saltwater, which can cause an intense stinging in the eye. In all cases with currently available goggles, the goggles must be removed in order for the user to wipe the eye or gain access to the eye.

In accordance with an aspect of the present invention, there is provided a device for protecting the eyes of a user from foreign elements including water, air, and debris, while also providing access to the user's eye during use of the device. The inventor has recognized a need for protective eyewear, particularly for use during kite boarding, and other similar water-sport activities, where there is a need to protect the eye from foreign elements, but where a sealed goggle structure precluding water from entering the eye upon being submerged under water is neither necessary nor ideal.

In one embodiment, the invention pertains to a protective eyewear device for wearing on a face of a user, which allows access to the eye without having to remove goggles from over the eyes. The embodiment allows eye access at each lateral end of the protective eyewear device. In a specific embodiment, the device includes a frame body that has a front side, a rear side, a bottom side, a top side, and a center portion. The frame has a first lateral end and a second lateral end and is configured such that the frame contacts the face except at the first and second lateral ends. The frame includes a transparent lens that is associated with the frame such that it shields the eyes of the user from a front side direction. The frame also includes a securing member associated therewith that holds the device onto the face such that said rear side of said frame is in contact with the face. The securing member attaches onto the frame at said first and second lateral ends of said frame in such a fashion as to create a lateral opening on both said first and second lateral ends of the frame. The device is configured to meet the face of the user such that the frame forms a gap between the face of the user and the device to allow access to the eye during use of the device. Moreover, the securing member is associated with the frame and is configured such that it does not obstruct this gap.

In a more specific embodiment, the back side of said frame is covered by a foam backing layer which is thicker at each said first and second lateral ends of said frame and gradually becomes thinner at the center of said frame. In another embodiment, the lens is removable and is attached onto the frame to form a space between the lens and the frame; and the lens is mounted onto said frame with flushmount or flat head stainless steel screws. In yet another embodiment, the securing member is comprised of a top band and a bottom band, wherein the top band connects to the first and second lateral ends proximate to or on the top side of the frame, and the bottom band connects to the first and second lateral ends proximate to or on the bottom side of said frame to secure the device to the face of the user. In an alternative embodiment, the securing member is comprised of a band body with diverging band ends on each side of the band body, wherein one end of the band attaches to the first lateral end of the frame and the other end of the band attaches to the second lateral end of the frame to secure the device to the face of the user.

In a more specific embodiment, the gap between the frame and the face of the user described above is between 1-6 centimeters depending on the facial structure of the user. In an even more specific embodiment, the gap is between 2-4 centimeters depending on the facial structure of the user.

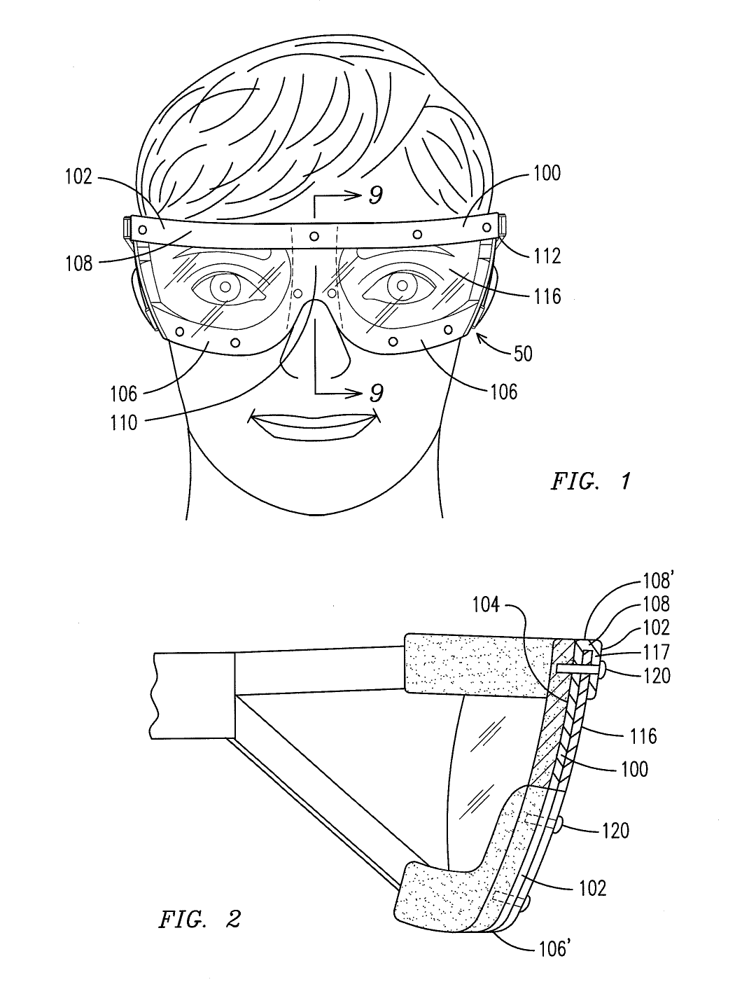

Turning to the drawings, FIG. 1 shows a front view of a protective eyewear device 50. The device includes a frame body 100 that has a front side 102 and a center portion 110 (dashed lines). Associated with the frame body 100 is a transparent lens 116. Also shown on this view is a first lateral end 112 of the frame body 100. FIG. 2 shows cross-sectional view on plane 9-9 from FIG. 1. As shown in this view, the frame body 100 has rear side 104, bottom portion 106 having a bottom side 106′, and top portion 108 having a top side 108′. This view shows that the lens 116 fits into a groove 117 defined in the top portion 108 of the frame 100. The lens 116 rests against the front side 102 of the frame at the bottom portion 106. The lens 116 is attached to the frame 100 for example by screws 120 (or other suitable fastener such as but not limited to, rivets, pins, staples, dowels etc. or alternatively via an adhesive thereby alleviating the need for fasteners, though fasteners and adhesives can be used together).

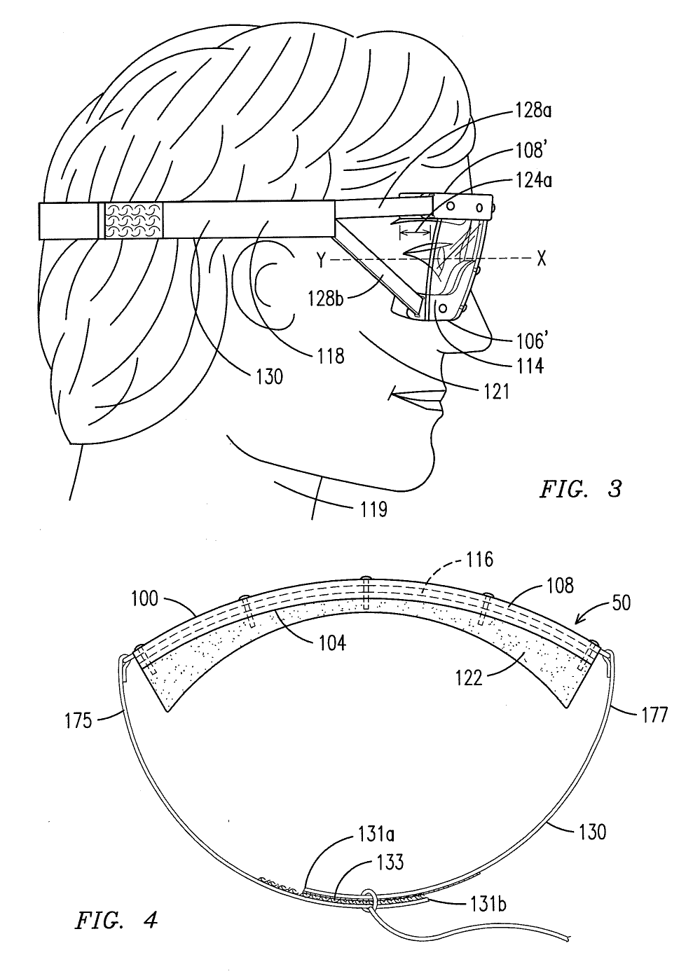

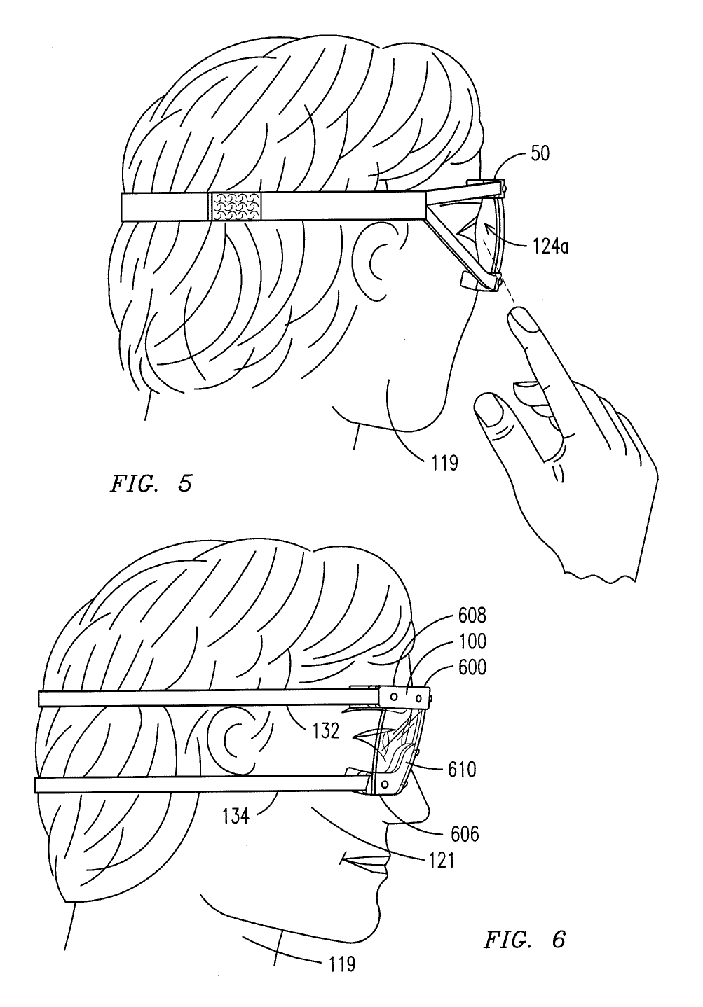

FIG. 3 shows a side view showing a second lateral end 114 of the protective eyewear device 50. Attached to the frame body 100 is a securing member 118. The securing member 118 has a band body 130 with a first divergent end 128a attaching proximate to the top portion 108 and a second divergent end 128b attaching proximate to the bottom portion 106. The first and second divergent ends 128a,b are provided so as to not obstruct the lateral gap 124a formed by the protective eyewear device 50 and face 121 of the user. FIG. 5 shows a side rear view which better illustrates the gap 124a. The gap 124a gives the user 119 access to wipe water from the eyes. However, the protective eyewear device 50 is sealed upon the user's face 121 at the top and bottom sides. Thus, the protective eyewear protects the eyes from turbulent splashing into the eyes, while preventing fogging experienced by convention goggles and enables the user 119 to clean their eyes without having to remove the protective eyewear device 50.

FIG. 4 shows a top view of the device embodiment 50. As can be seen in this view, the lens 116 dashed lines is shown fitting into the top portion 108 of the frame body 100 with screws 120. Those skilled in the art will appreciate that numerous other fasteners may be used when equipped with the teachings herein. Provided on the rear side 104 of the frame body 100 is a cushion pad layer 122. The band body 130 of the securing member 118 comprises opposing ends 175, 177. More specifically, as shown the band body 130 comprises a first band component 131a and a second band component 131b. The band components 131a,b are secured together by a hook and loop material 133.

As used herein, the term “proximate” when used with respect to the bottom side of the structure, (proximate to the bottom side) means somewhere between the centerline x-x′ axis (see FIG. 3) and the bottom side of a lateral end of the structure. “Proximate” when used with respect to the top side of the structure, (proximate to the top side) means somewhere between the centerline x-x′ axis (see FIG. 3) and the top side of a lateral end of the structure.

FIG. 6 shows a side view of an alternative embodiment of a protective eyewear device 600. According to this embodiment, the means for securing the device 600 to the face 121 of a user 119 includes a top band 132 and a bottom band 134. This is in place of the divergent band ends discussed in relation to FIG. 1. Important to this invention is that the top band 132 is attached to the frame 610 proximate to the top side 608 and the bottom band 134 is attached to the frame 610 proximate to the bottom side 606. This arrangement prevents the bands 132, 134 from obstructing the lateral gap 124a formed between the device 600 and the user's face 121.

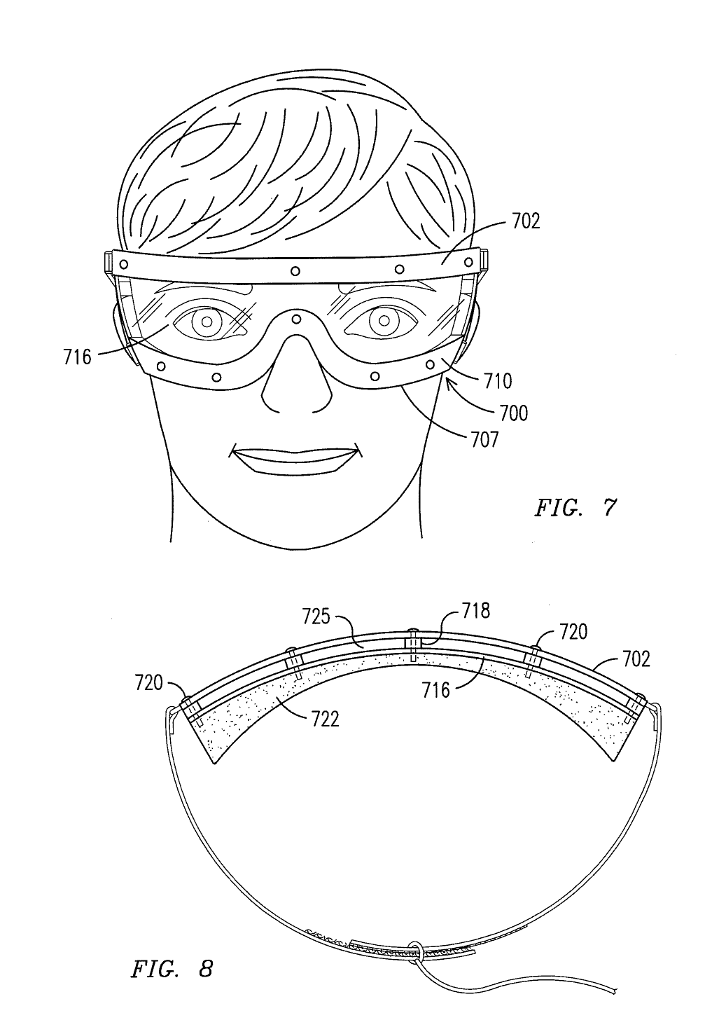

FIG. 7 shows a front view and FIG. 8 shows a top view of an alternative eyewear protective device embodiment 700. This embodiment 700 comprises a frame 710 that has a top portion 702 and a bottom portion 707 but lacks a center portion. The embodiment 700 includes a lens 716 that is attached to both top and bottom frame portions 702, 707. The top portion 702 is attached to the lens 716 by screws 720. Spacers 718 are provided between the top portion 702 which creates a ventilation space 725 between the lens 716 and the top portion 702. Note that the spacers 718 are optional and maybe included between the bottom portion (not shown) and the lens 716 as well. Associated with the back side of the lens 716 is a cushion pad layer 722.

The disclosures of the cited patent documents, publications and references are incorporated herein in their entirety to the extent not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims

1. A protective eyewear device for wearing on a face of a user, said device comprising:
a frame body comprising a front side, a rear side, a bottom portion having a bottom side, a top portion having a top side, and a center portion; a first lateral end and a second lateral end on each lateral side of said frame, the frame being configured such that the frame contacts the face except at the first and second lateral ends,
a transparent lens secured to said frame,
a securing member for holding said frame onto the face such that said rear side of said frame is in contact with the face; said securing member attaches onto said frame at said first and second lateral ends of said frame to create a lateral opening on both said first and second lateral ends of said frame forming a gap between the face of the user and the device to allow access to the eye during use of the device.

2. A device as in claim 1 wherein said rear side of said frame comprises a cushion pad which is thicker at each said first and second lateral ends of said frame and gradually becomes thinner at the center of said frame, wherein the cushion pad of the frame contacts the face except at the first and second lateral ends.

3. A device as in claim 1 wherein said lens is removable and is attached onto said frame to form a space between said lens and said frame; and said lens is mounted onto said frame with flushmount or flat head stainless steel screws.

4. A device as in claim 1 wherein said securing member is comprised of a top band and a bottom band, wherein said top band connects to said first and second lateral ends proximate to or on the top side of said frame, and said bottom band connects to said first and second lateral ends proximate to or on the bottom side of said frame to secure the device to the face of the user.

5. A device as in claim 1 wherein said securing member is comprised of a band body with diverging band ends on each side of said band body, wherein each band end attaches to said first and second lateral ends of said frame to secure the device to the face of the user.

6. A device as in claim 1 wherein said gap is between 1-6 centimeters depending on the facial structure of the user.

7. A device as in claim 4 wherein said gap is between 2-4 centimeters depending on the facial structure of the user.

8. A protective eyewear device for wearing on a face of a user, said device comprising:
a frame body comprising a front side, a rear side, a bottom portion having a bottom side, a top portion having a top side, and a center portion; a first lateral end and a second lateral end on each lateral side of said frame;
a transparent lens secured onto the frame, and wherein said lens rests in a groove defined within the top portion and rests on the front side of the frame at the bottom portion;
a cushion pad associated with the rear side of the frame, wherein the frame and cushion pad are configured such that the cushion pad contacts the face except at the first and second lateral ends; and
a securing member for holding said frame onto the face such that said rear side of said frame is in contact with the face; said securing member comprising a band body having opposing ends where the opposing ends each have a first diverging end that associates with said frame proximate to said top side and a second diverging end that associates with said frame proximate to said bottom side so as to create a lateral opening on both said first and second lateral ends of said frame, wherein the frame, cushion pad and securing member create a gap between the face of the user and the device to allow access to the eye during use of the device.

9. A device as in claim 8, wherein said gap is from about 1 centimeter to about 5 centimeters, or any 0.2 centimeter increments in between.

10. A device as in claim 8, wherein said band body comprises a first band body portion that associates with said first lateral end and a second band body portion that associates with the second lateral end, wherein said first and second band body portions engage via a hook and loop material.

11. A device as in claim 8, wherein said lens is secured to the frame by a plurality of fasteners.

12. A device as in claim 8, wherein said lens is secured to the frame via an adhesive.
